(19) United States
(12) Patent Application Publication
(10) Pub. No.: US 2003/0008000 A1
(43) Pub. Date: Jan. 9, 2003

(54) DNA vaccine using liposome-encapsulated plasmid DNA encoding for hemagglutinin protein of influenza virus

(76) Inventors: Jonathan P. Wong, Medicine Hat (CA); Leslie P. Nagata, Medicine Hat (CA)

Correspondence Address: RADER FISHMAN & GRAUER PLLC, LION BUILDING, 1233 20TH STREET N.W., SUITE 501, WASHINGTON, DC, 20036, US

(21) Appl. No.: 10/091,567

(22) Filed: Mar. 7, 2002

Related U.S. Application Data
(60) Provisional application No. 60/273,824, filed on Mar. 8, 2001.

(51) Int. Cl. A61K 48/00; A61K 9/127

(52) U.S. Cl. 424/450; 514/44

(57) ABSTRACT

DNA vaccination using plasmid encoding the hemagglutinin (HA) gene of influenza virus to induce long-lasting protective immunity against respiratory infection is disclosed. Efficacy of DNA vaccines is shown using a lethal influenza infection model in mice by employing liposomes as carriers. Mice immunized intranasally or intramuscularly with liposome-encapsulated pCI plasmid endcoding HA (pCI-HA10) are completely protected against an intranasal 5 LD₅₀ influenza virus challenge. Mice immunized with liposome-encapsulated pCI-HA10, but not naked pCI-HA10, by intranasal administration are found to produce high titers of serum IgA. The present invention shows that DNA vaccines encapsulated in liposomes are efficacious in inducing complete protective immunity against respiratory influenza virus infection.

### FIELD OF THE INVENTION

This invention relates to DNA vaccines which consists of plasmid DNA containing the hemagglutinin (HA) gene encapsulated in liposomes for inducing protective immunity against respiratory influenza virus infection

### BACKGROUND OF THE INVENTION

Despite recent advances in antiviral chemotherapy and vaccine development, infection and complications from influenza remain a leading cause of human mortality or morbidity globally. Influenza can be particularly fatal amongst the elderly and individuals with underlying medical problems, including immuno-compromised conditions. To date, vaccination using killed whole virus remains the most effective preventive measure against influenza.

Genetic vaccination using plasmid DNA represents an alternate and more desirable means of inducing protective immunity against viral infections. This new generation of vaccines provides many advantages over conventional live or killed vaccines. Unlike live or attenuated vaccines, DNA vaccines do not produce infection and therefore do not pose inherent safety concerns associated with live or attenuated vaccines. In addition, most subcellular vaccines only induce either humoral or cell-mediated immunity. DNA vaccines, on the other hand, can stimulate both humoral and cellular immune responses. These attributes make DNA vaccines attractive and promising candidates for treating respiratory viruses, including influenza.

Efficient expression of genes in plasmid DNA encoding the protective antigens requires the physical uptake of the plasmid by the target cells. Although there is ample evidences which suggest that naked plasmid DNA injected directly into the muscles can express the gene of interest and induce protective immunity, its ability to be taken up by mucosal-associated lymphoid tissues and to induce mucosal immunity has not been well documented. Furthermore, needle injection of the DNA vaccines in muscles in humans can be painful and may present health-related safety concerns.

Since influenza pathogens primarily infect and multiply in the lower respiratory tract, delivery of DNA vaccines to the respiratory tract has the potential to induce a focussed and long-lasting protective immunity in the lungs. In addition, the lymphoid tissues found in the large mucosa surfaces in the respiratory tract can enable mucosal immunity to be induced, thereby helping to prevent virus attachment of the lung epithelial cells and strengthening the overall immune defence against the infectious virus particles. Accordingly, development of a DNA immunization strategy that could induce protective mucosal immunity can be invaluable in reducing morbidity and mortality associated with infections caused by these pathogens.

It has been recognized that a major problem with the current influenza vaccines is that they may be ineffective against new variants of the viruses resulting from genetic changes such as antigenic drifts in the HA protein or antigenic shifts to another HA subtypes. Accordingly, it is desirable to introduce DNA vaccines capable of inducing long lasting immunity against the HA protein.

### Literatures of General Interest

Centers for Disease Control. Prevention and control of influenza:recommendations of the Immunization Practices Advisory Committee. Morbid And Mortal. Weekly Report, 1992; 41 (RR-9):1-5.

Pardoll, D R, Beckering, A M. Exposing the immunology of naked DNA vaccines. Immunity 1995; 3:165-169.

Cox, G J M, Zamb, T J, Babiuk, L A. Bovine herpesvirus I:Immune responses in mice and cattle injected with plasmid DNA. J. Virol. 1993; 67:5664-7.

Donnelly, J J, Ulmer, J B, Shiver, J W, Liu, M A. DNA vaccines. Ann. Rev. Immunol. 1997; 15: 617-648.

Ulmer, J B, Deck, R R, DeWitt, C M, Friedman, A, Donnelly, J J, Liu, M A. Protective immunity by intramuscular injection of low doses of influenza virus DNA vaccines. Vaccine 1994; 12: 1541-4.

Doe, B, Selby, M, Barnett, S, Baenziger, I, Walker, C M. Induction of cytotoxic T lymphocytes by intramuscular immunization with plasmid DNA is facilitated by bone marrow-derived cells. Proc. Natl. Acad. Sci. U.S.A. 1996; 93:8578-83.

Townsend, J R, McMichael, A J, Carter, N P, Huddleston, J A, Brownlee, G G. Cytotoxic T cell recognition of the influenza bucleoprotein and hemagglutinin expressed in transfected mouse L cells. Cell 1984; 39:13-25.

Tabor S and Richardson C C. A bacteriophage T7 RNA polymerase/promoter system for controlled exclusive expression of specific genes. Proc. Natl. Acad. Sci. USA, 1985; 82:1074-1078.

Long, M C, Jager, S, Mah, D C W, JeBailey, L, Mah, M A, Masri, S A, Nagata, L P. Construction I) and characterization of a novel recombinant single-chain variable fragment antibody against western equine encephalitis virus. Hybridoma 2000; 19:1-13.

Wheeler, J J, Palmer, L, Ossanlou, M, MacLachlan, T, Graham, R W, Zhang, Y P, Scherrer, P, Cullis, P R. Stbilized plasmid-lipid particles:construction and characterization. Gene Ther. 1999; 6:271-281.

Wong, J P, Stadnyk, L L, Saravolac, E G. Enhanced protection against respiratory influenza A infection by liposome-encapsulated antibody. Immunology 1994; 81:280-284.

McCluskie, M J, Davies, H L. Novel strategies using DNA for the induction of mucosa immunity. Critic. Rev. in Immunol 1999; 19:303-329.

Mahato, R I, Anwer, K, Tagliaferri, F, Meaney, C, Leonard, P, Wadhwa, M S, Logan, M, French, M, Rolland, A. Biodistribution and gene expression of lipid/plasmid complexes after systemic administration. Human Gene Ther. 1998; 9:2083-2099.

### SUMMARY OF THE INVENTION

This invention is directed to liposome-encapsulated deoxyribonucleic acid (DNA) vaccines using plasmid encoding the hemagglutinin (HA) gene of the influenza virus. More specifically, the present invention is focussed on the induction of strong and specific mucosal immunity in the respiratory tract through pulmonary delivery of such DNA vaccine encapsulated in liposomes by intranasal instillation or aerosol inhalation.

An object of the present invention is to provide liposomes as vaccine carriers. Liposomes can effectively deliver charged molecules such as DNA intracellularly and to the sites of infection. More particularly, the present invention focuses on a novel liposome encapsulated DNA vaccines using plasmid encoding the HA gene of the influenza virus.

In accordance with one aspect of the present invention, there provides a DNA vaccine against influenza which comprises of a plasmid carrying a gene encoding the HA protein and its delivery to site of infection by encapsulation of the vaccine in liposomes.

In accordance with another aspect of the present invention, there provides a transfer vector comprising a pCI-HA10 plasmid adapted for transformation of a microorganism host.

In accordance with a further aspect of the present invention, there provides a process of cloning a pCI-HA10 plasmid comprising re-amplifying HA gene with primers; ligating HA gene into a pCI vector; transforming said the pCI vector into competent E. coli cells; and transcribing and translating pCI-HA10 clones.

In accordance with yet another aspect of the present invention, there provides the use of a liposome-encapsulated DNA vaccine encoding the HA gene to prevent and/or treat respiratory influenza virus infection.

### BRIEF DESCRIPTION OF THE DRAWINGS

The present invention will now be described with reference to the following drawings:

FIG. 1 is a schematic representation of pCI-HA10 depicting map of plasmid, cloning and restriction sites and location of HA insert.

FIG. 2 is an autoradiograph of HA product by SDS-PAGE. The PCI-HA10 plasmid was transformed by competent E.coli DH5α cells. In vitro transcription/translation of the HA product was performed in the presence of canine microsomal membranes and [³⁵S]-methionine, and analysed by SDS-PAGE and autoradiography. Lane (a) molecular weight rainbow marker, (b) no DNA, (c) positive control pT75-HA16, (d) pCI vector, no insert, (e-g) pCI-HA10, (h) luciferase translation kit control.

FIG. 3 shows the efficacy of intranasally administered liposome-encapsulated pCI-HA10 against influenza virus in mice. Mice intranasally immunized with one primary and 3 booster doses of liposome-encapsulated pCI-HA10 (Lipo pCI-HA10), naked pCI-HA10 or liposome-encapsulated pCI. At one week post final immunization boost, the mice were intranasally challenged with 5 LD₅₀ of virus. The survival rates were monitored daily.

FIG. 4 illustrates the efficacy of intramuscularly injected naked and liposome-encapsulated pCI-HA10 to protect mice against respiratory lethal influenza virus challenge.

FIG. 5 shows the IgA levels of mice immunized intranasally with naked and liposome-encapsulated pCI-HA10. Each mouse received one primary and 3 booster doses of liposome-encapsulated (Lipo pCI-HA10) or naked pCI-HA10. At one week post final boost, the animals were tail bled and IgA titers in the serum samples were determined by IgA HA ELISA.

### DETAILED DESCRIPTION OF THE INVENTION

One of the most attractive features of genetic vaccination is the possibility and flexibility to clone one or more uniquely designed gene sequences of protective antigens into plasmids. This allows for rational designs of plasmid DNAs to be used in vaccination program against the current strains and subtypes of influenza viruses. Due to the ease of specific gene designs and scale-up procedures afforded by molecular biology, DNA vaccination offers many advantages over conventional live, killed or attenuated vaccines such as stability, safety and design.

Genetic vaccination is a promising and exciting means of inducing both humoral and cellular protective immunity in vivo. Antibodies produced against the antigenic domains encoded by the vaccine provide humoral immunity, but, in addition, the protein is synthesized within a cell, thereby potentially generating cell mediated immunity activation. DNA vaccines also provide the opportunity to specifically encode protective proteins which elicit protective immunity without introducing additional molecules into the body. Moreover, mass production of highly purified vaccines is easily achieved with DNA vaccines.

Naked plasmid DNA injected directly into muscles of animals are taken up and expressed by cells in vivo and has been shown to induce both humoral and cellular immune responses against the encoded antigens. Intramuscular injection of naked plasmid DNA containing the HA protein is also capable of inducing both cellular and humoral immune responses to influenza virus.

In the present invention, it has been found that liposome-encapsulation of the plasmid DNA enhances the effectiveness of the vaccine. Liposomes are biologically similar to cell membranes, and hence, they can interact and fuse with the cell membranes and deliver large, charged molecules, such as DNA, into the cell. Lipids, which are the basic components of liposomes, can entrap DNA molecules while forming a membrane vesicle-like structure. Liposomes also protect the DNA from in vivo dilution and endonuclease degradation, and are non-toxic, biodegradable, and relatively non-antigenic in mammalian systems.

Influenza virus has been identified to contain two major surface proteins, namely HA and neuraminidase. A third protein, the matrix protein, forms a bridge between the surface proteins and the nucleoprotein core in viral assembly. In the present invention, DNA vaccines against influenza virus infection are described. This vaccine is based on the gene encoding the HA protein and on the use of liposomes to enhance HA delivery and expression. Hemagglutinin elicits a strong, neutralizing antibody response against the influenza virus. However, HA undergoes mutations regularly within antigenic domains resulting in antigenic drifts as well as shifts. The use of DNA vaccines offer advantages over conventional attenuated vaccines in that minor changes in the HA gene can be easily introduced through recombinant techniques. In addition, a number of HA variants can be incorporated in the same plasmid DNA to provide cross protection against HA mutants. Liposome encapsulated DNA vaccine of the present invention is capable of inducing long lasting immunity against the HA protein.

The process of cloning and encapsulating the HA gene within liposomes in accordance with the present invention is described in more details below.

Hemagglutinin gene are cloned by the following procedure:

(1) Excision and expression of HA gene construct from influenza virus;

(2) Re-amplification of HA gene with primers;

(3) Ligation of HA gene into a plasmid vector;

(4) Vector transfer; and

(5) HA clone transcription and translation.

### MATERIALS AND METHODS

### Cloning and Characterization of HA Gene

The original HA construct from influenza A/PR/8/34, P8H is known. The HA was excised with HindII and BamHI followed by subcloning into pT7-6 to give pT76-HA16, which expresses the HA gene from a T7 promoter. The HA gene was re-amplified from the template pT76-HA16 clone using the following primers: HAXba5′, sense (5′TATCTAGACAAAAGCAGGGGAAAATAAAACAACCAAAATG 3′); HANot3′, antisense, (5′AAGTCATAGCGGCCGCAAGGGTGTTTTTCCTCATATTTCT 3′). The Xba I and Not I sites in HAXba5′ and HANot3′, respectively, are in italics. Amplification of the HA gene was At accomplished by PCR (polymerase chain reaction) using the GeneAmp XL PCR™ kit with rTth DNA polymerase (Perkin Elmer, Foster City, Calif.) followed by column purification using the QIAquick PCR™ purification kit (Qiagen, Valencia, Calif.). The amplified HA gene was further digested with Xba I and Not I to create the respective sites at the 5′ and 3′ ends. The HA gene was then ligated into the pCI vector (Promega Corporation, Madison, Wis.) at Xba I and Not I restriction sites, and transformed into competent E. coli DH5α cells (Gibco/BRL, Bethesda, Md.). The pCI-HA10 clone was identified as containing the full-length HA gene, by restriction mapping and DNA sequencing (data not shown). In vitro transcription and translation of the pCI-HA10 clone were performed using the TNT coupled system (Promega Corporation) and canine microsomes (Promega) as described by Long et al., supra.

Bulk preparations of pCI-HA10 were prepared with the Endofree Plasmid Mega and Giga™ kits (Qiagen) following manufacturer's directions and analysed by restriction enzyme digests. Only plasmid DNA of greater than 90% purity was used for liposome encapsulations and animal immunizations.

### Liposome Encapsulation of pCI-HA10

Cationic liposomes used for the encapsulation of pCI-HA10 were prepared using a modification of a procedure described by Wheeler et al., suira. Briefly, liposomes consisting of 7% 1,2-dioleoyl-3-dimethylammonium chloride (DODAC, Avanti Polar Lipid Inc., Alabaster, Ala.), 78% 1,2-dioleoyl-sn-glycerol-3-phosphoethanolamine (DOPE, Avanti Polar Lipid Inc.) and 15% polyethylene glycol C8 (PEG₂₀₀₀C₈CER, Norther Lipid Inc., Vancouver, BC) were used at 10 mg/ml concentrations. The lipid film was formed at 50° C. using a rotaevaporator (Buchi Rotavapor R110, Brinkman, Rexdale, ON), and then incubated at 50° C. for 2 h under vacuum. The lipid film was reconstituted with distilled water and 1M β-octylglucanopyranoside (OGP, Sigma, Mississauga, ON) detergent at 20% of the total preparation volume. The plasmid DNA was next added to the lipid film at a concentration of 400 μg DNA/ml of 10 mg/ml. The reconstituted preparation was transferred into dialysis tubing (Spectra/Por, MWCO: 12-14,000, Spectrum Laboratories Inc., Rancho Dominguez, Calif.) and dialyzed in 1X HEPES buffer solution (150 mM NaCl, 20 mM Hepes, pH 7.4) at 23° C. for 15 h. The free, non-encapsulated DNA was removed from encapsulated DNA on a DEAE Sepharose CL-6B (Sigma) anion exchange column. Encapsulation recovery ranged from 38.0% to 57.0% (data not shown). The liposomes preparations were concentrated using Aquacide II™ (Calbiochem, La Jolla, Calif.) and polyethylene glycol MW 10,000 (Sigma) and then dialyzed in 1× HEPES for an additional 2 h at 23° C. Particle size analysis of liposome encapsulated DNA was performed using a Zetqasizer 3000™ (Malvern Instruments, Point Roberts, Wash.).

### DNA Vaccination of Mice

Six week old female BALB/c mice were obtained from the mouse breeding colony at the Canadian Defence Research Establishment Suffield (DRES). The mice were immunized with naked or liposome-encapsulated plasmid DNA using intramuscular (IM) or intranasal (IN) routes of administration. For intramuscular injection, mice were anaesthetized with ketamine:xylazine (50 mg/kg: 50 mg/kg body weight) into the hind leg. A small incision was made exposing the quadriceps muscle, and 50 μl of 1 mg/ml DNA preparation was injected slowly. The incision was then sutured. For intranasal administration, mice were anaesthetized with sodium pentobarbital (50 mg/kg body weight) by intraperitoneal injection. When the animals were completely unconscious, 50 μl of 0.4 mg/ml DNA preparations were administered gently into one of the nostrils with a micropipettor. To avoid swallowing of the plasmid into the stomach, the intranasal dosing was given when the animals were completely anaesthetized. The applied volume was naturally inhaled into the lungs. Both IM and IN groups received 1-3 additional boosts of DNA, given four weeks apart. One week after each boost, approximately 200 μl of blood was collected via tail bleed and analysed for anti-HA IgA by enzyme-linked immunosorbent assay (ELISA).

### Virus Challenge of Immunized Mice

For animal virus challenge studies, a mouse-adapted strain of influenza A/PR/8/34 (H1N1) was used. This strain was obtained by at least four blind passages in mice using egg-propagated virus (ATCC, Parklawn, Md.) as the initial inoculums. The passaging and propagation of this mouse-adapted strain of influenza virus had been previously described in Wong et al., supra.

For the vaccine efficacy study, mice immunized with the DNA vaccine were challenged with the virus as described below. A week following the last booster dose, the animals were anaesthetized with sodium pentobarbital (50 mg/kg body weight, i.p.). When the mice were completely anaesthetized, they were inoculated with 50 μl of the egg-propagated virus by intranasal instillation into the nostrils. The challenge infectious dose was 5 LD₅₀ unless otherwise stated. At 14 days of post infection, the number of surviving mice in each of the control and test groups was recorded. The number of mice in each control and test group used was 10 per group.

### HA Indirect ELISA

Mouse-adapted, egg-propagated influenza virus A/PR/8/34 was purified from allantoic fluid by sucrose gradient purification method. Briefly, the influenza virus was precipitated from allantoic fluid with 7% polyethylene glycol and 2.3% sodium chloride with gentle stirring for 15 hat 4° C. The virus particles were collected by centrifugation at 10,000 g for 30 min at 23° C. The pellet was resuspended in phosphate buffered saline (PBS) and layered onto a 20-60% sucrose gradient. After ultracentrifugation at 100,000 g for 4 h at 4° C., the virus band was isolated and dialyzed in 0.9% saline for at least 3 h at 23° C . Purified influenza virus was assayed by titration with monoclonal anti-influenza virus type A (HA) antibody (Biodesign International, Saco, Me.) to determine the optimum antigen concentration for ELISA. Dilution of 1/20 of the purified influenza virus antigen and coating buffer (15 mM sodium carbonate, 35 mM sodium bicarbonate, 0.02% (w/v) sodium azide, pH 9.6) were used to coat the Nunc Maxisorb flat bottomed 96-well plates (Gibco BRL, Gaithersburg, Md.). The plates were sealed and incubated at 4° C. for 15 h. The plates were then washed 5 times with 0.1% BSA, 1% Tween 20 in PBS, blocked with of 2% BSA, 1% Tween 20 in PBS for 1 h at 37° C. and incubated with serial dilutions of test mouse sera. After 1 h incubation at 37° C. and washing as described above, the bound antibody was detected by peroxidise-labelled goat anti-mouse IgA, (KPL, Gaithersburg, Md.). The peroxidise activity was measured using 2,2′-azino-di[3-ethyl-benzthiazoline sulfonate], (KPL) as a substrate and measured at 405 nm after 20 minutes of incubation at 23° C.

### Statistical Analysis

The survival rates of the test vaccinated and control groups were compared by the Mann-Whitney unpaired non-parametric one-tailed test (In Stat, version, Graph-Pad software, San Diego, Calif.).

### RESULTS

### Cloning and Expression of HA Gene

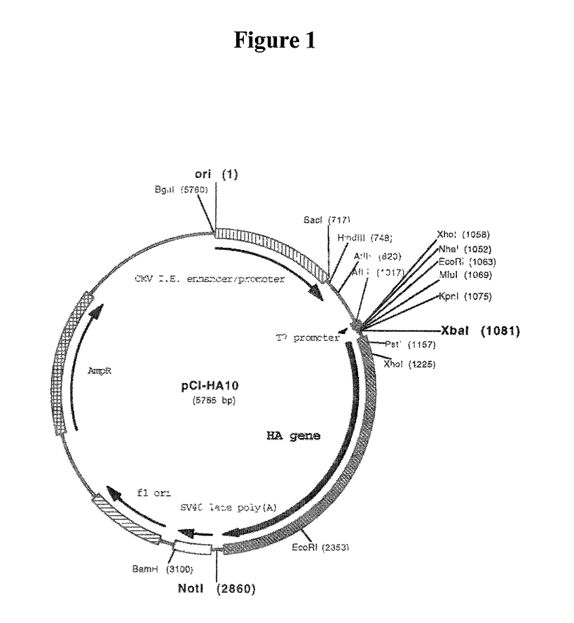

The HA gene used in this study was originally cloned and expressed in the pT76 vector as described previously by Townsend et al. and Tabor et al., supra. The HA was amplified by polymerase chain reaction, and the PCR product digested with XbaI and EcoRI, and the fragments were cloned in pCI vector using T₄ DNA ligate. The resultant construct, referred to as pCI-HA10, is shown schematically in FIG. 1.

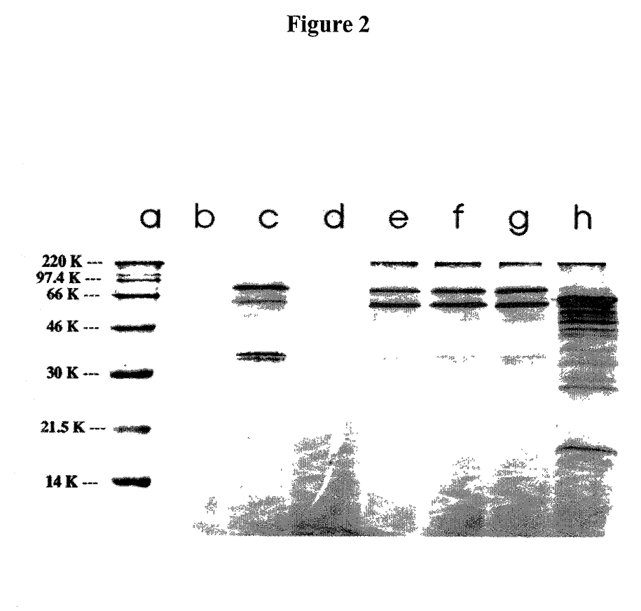

The pCI-HA10 plasmid was transformed into competent E.coli DH5α cells. In vitro transcription/translation of the HA product was performed in the presence of canine microsomal membranes and [³⁵S]-methionine, and analysed by SDS-PAGE and autoradiography (FIG. 2). The two bands of 82K and 66K appear to represent the glycosylated and unglycosylated forms of HA protein. The expression of HA by pCI-HA10 was then confirmed by western blot using a specific anti-H1 hemagglutinin monoclonal antibody (results not shown).

### Efficacy of DNA Vaccination

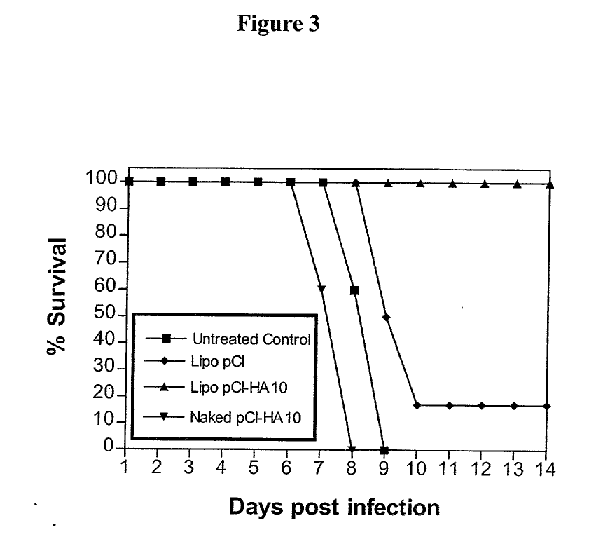

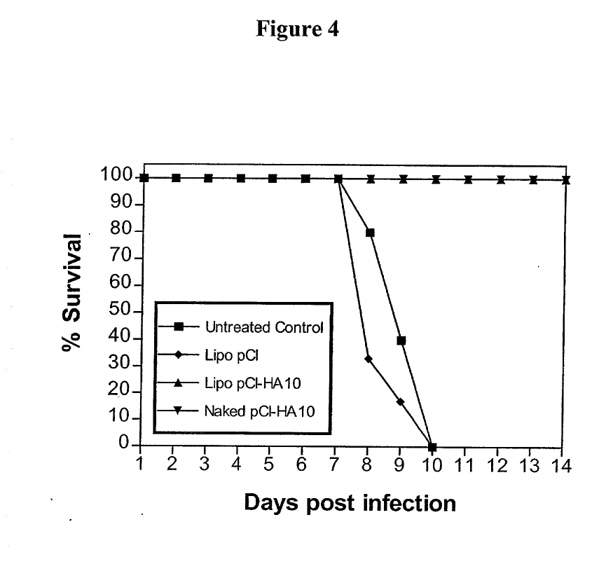

The efficacy of naked and liposome-encapsulated pCI-HA10 plasmids to protect animals against lethal challenge of influenza virus by intranasal and intramuscular administrations is shown in FIGS. 3 and 4. Non-immunized mice succumbed to the influenza infection at as early as 7 days post infection, and all animals were dead by day 9. All mice which received intranasal immunization with naked unencapsulated pCI-HA10 also succumbed to the infection, with no increase in survival rate nor survival time (FIG. 3). In contrast, mice immunized intranasally with liposome-encapsulated pCI-HA10 were found to be completely protected with 100% survival rate (p<0.01 vs. control or naked pCI-HA10 group).

When the pCI-HA10 DNA was administered by intramuscular injection, both liposome-encapsulated and naked pCI-HA10 plasmid were shown to provide complete protection against the virus challenge (FIG. 4). In contrast, liposome-encapsulated pCI without the HA insert provided little or no protection.

### IgA Titers in Sera

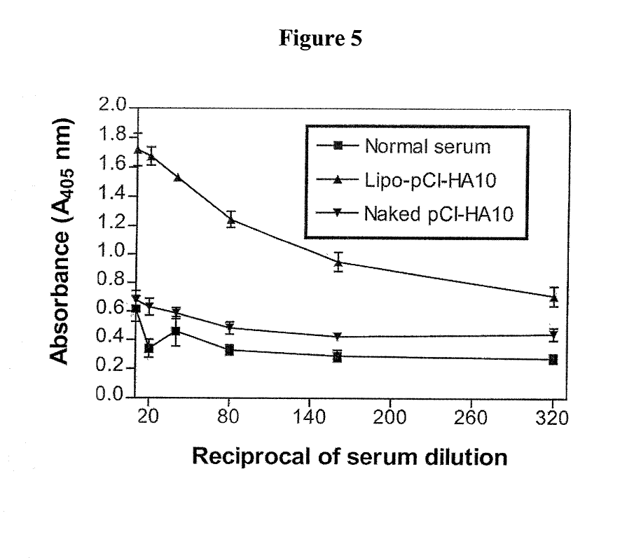

Specific IgA titers in serum samples of mice in the various immunized groups were determined by indirect ELISA assay. Mice immunized with liposome-encapsulated pCI-HA10 by intranasal route were found to contain high titers of specific IgA in the sera, while those immunized with naked unencapsulated pCI-HA10 produced only marginally detected levels (FIG. 5). Specific IgA antibody was not detected in the serum samples from non-immunized mice or from mice immunized with liposome-encapsulated pCI without the HA insert.

Immunization of mice using intramuscular injection of naked or liposome-encapsulated pCI-HA10 did not result in any significantly high levels of specific HA IgA. Up to 3 booster injections were intramuscularly given, but no increase in IgA levels was observed (results not shown).

### DISCUSSION

Findings of the present invention suggest the use of vaccine carriers such as liposomes can offer many significant advantages. When intranasal immunization of animals was carried out using pCI-HA10 encapsulated in liposomes, there was a significant enhancement in vaccine efficacy as well as induction of strong mucosal immunity against the expressed antigen. Intranasal immunization using naked unencapsulated pCI-HA10 did not provide any significant protection, and did not result in the induction of mucosal immunity. Although naked DNA administered by intramuscular injection can induce strong systemic cellular and humoral immune responses, it is considered to be poor inducer of mucosal immunity.

When plasmid DNA is administered into the respiratory tract, liposomes can facilitate the uptake and transport of the plasmid DNA into the induction and effector sites in the bronchus-associated lymphoid tissues, or the nasal-associated lymphoid tissues. The delivery of the plasmid DNA by liposomes to these sites can result in the induction of protective mucosal immunity in the mucosa surfaces in the respiratory tract. This may account for the observation that liposome-encapsulated pCI-HA10 induced strong mucosal IgA response when it is administered into the respiratory tract but did not when injected directly into the muscles. Similarly, intranasal immunization with naked pCI-HA10 did not elicit any detectable mucosal IgA response. The inability of intranasal immunization with naked plasmid DNA to induce mucosal immunity is also reported by Pardoll et al., supra. These findings support the importance of using liposomes as vaccine carriers to the mucosal surfaces.

Mucosal vaccination using liposome-encapsulated plasmid DNA could also be very important in eliciting protective immunity at site distant from site of vaccine administration. According to the present invention, intramuscular immunization with naked and liposome encapsulated pCI-HA10 provided complete protection. This may suggest that induction of mucosal immunity is not essential for protective immunity against influenza. However, the dose of pCI-HA10 by intramuscular injection required to achieve complete protection was 2.5-fold higher than intranasal immunization. This may suggest mucosal immunity may contribute to the overall protective immunity against respiratory influenza infection.

Vaccination using plasmid DNA encapsulated in liposomes may offer other distinct advantages over naked plasmid DNA. It has been reported that liposomes can protect the plasmid DNA against nuclease degradation in the lungs or the in the serum and therefore increasing body retention times and enhancing immunological responses. Secondly, the use of liposomes is known to facilitate uptake of the plasmid DNA by the antigen-presenting cells, thereby increasing transfection efficiency. Genetic vaccination strategy using plasmid DNA encapsulated in liposomes represents an important advance in the induction of protective immune responses against influenza virus infection.

It is to be understood that the embodiments and variations shown and described herein are merely illustrative of the principles of this invention and that various modifications may be implemented by those skilled in the art without departing from the scope and spirit of the invention.

We Claim:

1. A deoxyribonucleic acid (DNA) vaccine against influenza virus infection comprising a plasmid carrying a gene encoding hemagglutinin (HA) protein, said DNA vaccine being encapsulated in liposomes.

2. A vaccine as claimed in claim 1 directed to the induction of mucosal immunity for treating respiratory influenza virus infection.

3. A vaccine as claimed in claim 2 targeting the mucosal surfaces in the respiratory tract of a host.

4. A transfer vector comprising a pCI-HA10 plasmid adapted for transformation of a microorganism host.

5. A process of cloning a pCI-HA10 plasmid comprising:
(1) re-ainplifying hemagglutinin (HA) gene with primers;
(2) ligating HA gene into a pCI vector;
(3) transforming said vector into competent E. Coli cells; and
(4) transcribing and translating pCI-HA10 clones.

6. Use of a liposome-encapsulated DNA vaccine encoding the hemagglutinin (HA) gene to treat respiratory influenza virus infection.

7. Use of a liposome-encapsulated DNA vaccine as claimed in claim 6, wherein said vaccine is administered intranasally.
